(19) United States
(12) Patent Application Publication
(10) Pub. No.: US 2020/0101185 A1
(43) Pub. Date: Apr. 2, 2020

(54) A METHOD FOR DISINFECTION AND CLEANING OF AT LEAST ONE PART OF A DEVICE INTENDED FOR RECYCLING OF WATER

(71) Applicant: ORBITAL SYSTEMS AB, MALMÖ (SE)

(72) Inventors: Gustav Rehn, Lund (SE); Stephan Eskilander, Lund (SE)

(21) Appl. No.: 16/621,406

(22) PCT Filed: Jun. 12, 2018
(86) PCT No.: PCT/SE2018/050610

§ 371 Date: Dec. 11, 2019

(30) Foreign Application Priority Data
Jun. 16, 2017 (SE) 1750768-2

(51) Int. Cl. A61L 2/18; C02F 1/00; C02F 1/68; C02F 1/50; E03B 1/04; E03C 1/04

(52) U.S. Cl. CPC A61L 2/18; C02F 1/001; C02F 1/686; C02F 1/50; E03B 1/042; E03B 2001/045; C02F 2201/006; C02F 2303/20; C02F 2303/22; C02F 2305/04; C02F 2307/06; E03C 1/0408

(57) ABSTRACT

The present invention describes a method for disinfection and cleaning of at least one part of a device intended for recycling of water, said method involving flowing lactic acid, citric acid and/or an enzyme based or surfactant based solution into the part intended to receive the disinfection and cleaning.

### FIELD OF THE INVENTION

The present invention relates to a method for disinfection and cleaning of a water recycling device.

### TECHNICAL BACKGROUND

There are different water recycling devices known today. One example is shown in WO 2013/095278 where there is disclosed a hybrid device for a recirculation shower, allowing purification and either recycling of water or discarding of water. The hybrid device comprises a recirculation loop, a filter system with a pre-filter and a nano-filter, at least one filter quality sensor, and the device is arranged to redirect the water from recirculation to drainage when the at least one filter quality sensor indicates the need thereof.

The present invention is directed to a disinfection and cleaning method suitable for water recycling devices. The water recycling devices may be of any application type, such as e.g. recirculating showers, recirculating dishwashers or recirculating washing machines. The method is especially suitable for water recirculating devices used domestically, but also other usage is totally possible. Examples are recirculating showers systems used in hotels and bathhouses. Fact is that any type of such system, used industrially or domestically, is of interest for the method according to the present invention.

Moreover, it should be noted that the common feature of the water recirculating systems intended above is the possibility of recirculating water to be recycled and reused. Water being contaminated, or a liquid fraction containing contaminants, however, should be flowed to a drain. Also this fraction may be recycled in another system or collected depending on the kind of system and type of contaminants.

The present invention is directed to providing an optimal disinfection and cleaning method for water recirculating systems mentioned above.

### SUMMARY OF THE INVENTION

The latter stated purpose above is achieved by a method for disinfection and cleaning of at least one part of a device intended for recycling of water, said method involving flowing lactic acid, citric acid and/or an enzyme or surfactant based solution into the part intended to receive the disinfection and cleaning.

In relation to the method according to the present invention it should be mentioned that the method involves both disinfection implying killing or inactivating bacteria and microorganisms, and also cleaning implying to remove dead or inactivated bacteria and microorganisms from tubing etc. The latter step can be seen as an antifouling step, so that the material is possible to be flush out from the water recirculating system or device so such material is not accumulated in the system.

### BRIEF DESCRIPTION OF THE DRAWINGS

FIG. 1 is an exemplary illustration of a water circulating device according to an embodiment of the present invention; and

FIG. 2 is a schematic diagram depicting disinfecting, cleaning and descaling effects according to an embodiment of the present invention.

### DETAILED DESCRIPTION

Below there is disclosed and discussed different embodiment of the method according to the present invention.

According to one specific embodiment of the present invention, the method involves at least flowing lactic acid into the part intended to receive disinfection treatment. Lactic acid may be used alone or in combination with other substances. According to one specific embodiment, the method involves flowing both an antibacterial agent for the disinfection and also an antifouling agent for the cleaning into the part intended to receive the disinfection and cleaning. Also in this case the antibacterial agent may be lactic acid. Furthermore, the flowing of the antibacterial agent and antifouling agent may be performed simultaneously or sequentially.

The substances intended to be used in the method according to the present invention have the advantage of being environmentally friendly and biologically degradable to a large extent. As the disinfection and cleaning method according to the present invention is intended to be used in devices intended to save water and energy the aspect above is of great importance. The usage of environmentally friendly and biologically degradable substances which has a very low negative impact on water and energy loss and use of chemicals is of course preferable in water recirculating systems, and in fact also in other systems where water is used and some amounts are wasted.

Moreover, both lactic acid and citric acid, and also several enzyme or surfactant based substances are easy to use and inexpensive.

The device intended for method comprises a flow path for recycled water. Other units of the device are a fresh water inlet, a recirculation water inlet and a recirculation water outlet, a user outlet, a pump, sensors, valves, a heater and a filter. According to one specific embodiment of the present invention the method involves flowing lactic acid, citric acid and/or an enzyme based or surfactant based solution into at least part of the flow path for recycled water. One part of special interest for the method is the user outlet. In this part and the tubing in close proximity to the user outlet there is an evident risk for growth of bacteria and microorganisms. Therefore, this part is of special interest for the method according to the present invention. According to yet another embodiment in line with this, the process order subsequent to the filter is the heater and then finally the user outlet and wherein flowing of lactic acid, citric acid and/or an enzyme based or surfactant based solution is performed into at least the user outlet of the device. Also in this case only lactic acid may be used, but also in combination with an enzyme based and/or surfactant based solution.

According to yet another embodiment, the flowing of lactic acid, citric acid and/or an enzyme or surfactant based solution is provided from the filter into the system and thus into all of the system arranged subsequent to the filter, also comprising the heater and the user outlet.

The provision of the substance(s) into the system may be provided by use of different means. According to one embodiment, a disposable filter unit is replaced with a cartridge containing the lactic acid, citric acid and/or an enzyme or surfactant based solution for allowing for performing the disinfection treatment. The cartridge is of course also a disposable to be used for the disinfection and cleaning of the system. The time point for performing the disinfection and cleaning may vary, but is intended to be performed at a decided time. This can be set by the system as such, e.g. when sensors are giving data to the processor that it is time for performing a disinfection and cleaning cycle. In such a case the system will inform a user that the method should be performed. Also a set certain use period (weeks, months) or a number of use cycles may be the parameters deciding when the method for disinfecting and cleaning should be performed.

As mentioned above, the method according to the present invention may be performed by use of a cartridge or cassette. According to yet another embodiment, the filter of the system is a disposable filter unit also comprising a dosing capsule containing lactic acid, citric acid and/or an enzyme or surfactant based solution and wherein the dosing capsule discharges the content when the filter is pressurized. The dosing of the lactic acid, citric acid and/or an enzyme or surfactant based solution may be performed during a time sequence in the beginning of usage of a new filter or may in fact be dosed based on a slow release technique so that the substance(s) are added continuously into the system at a suitable amount.

Another possible means for adding the substance(s) used in the method according to the present invention is dosage container. Therefore, according to one embodiment of the present invention, the lactic acid, citric acid and/or an enzyme or surfactant based solution is provided in a dosage container which content is poured into a drainage and thus into a recirculation loop to be flowed into the filter, the heater and the user outlet. Also in this case the substance(s) reached the user outlet and disinfects and cleans also this part. In this case, then the disinfection and cleaning is performed as a first cycle, such as during the first 10 minutes, when a new disposable filter unit is placed into the system.

As mentioned above, the water recirculating device may be of different type. Examples are showers, both domestically or at hotels or bathhouses, washing machines, or dish washers etc. According to one embodiment, the device intended for usage and recycling of water is a recirculating shower. As should be evident, when the disinfection and cleaning method is being performed, then regular use is not performed at the same time.

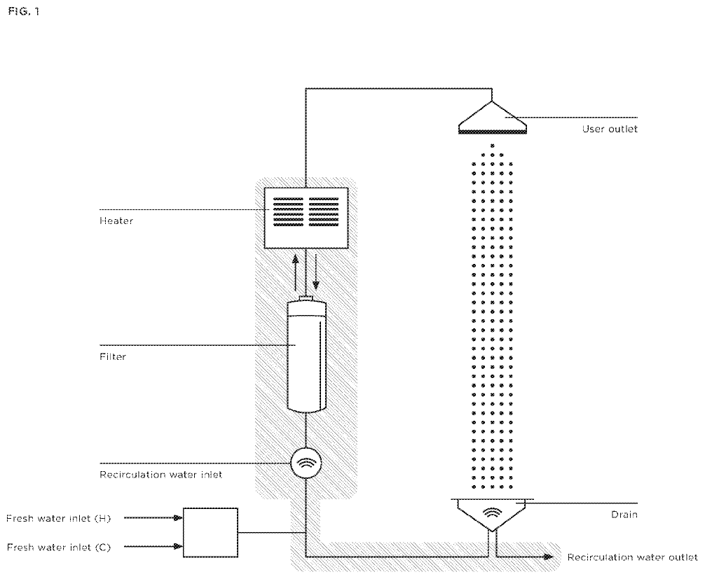

In FIG. 1 there is shown a water recirculating device, in this case a recirculating shower, in which the method according to one embodiment of the present invention may be used. As seen in FIG. 1, the device comprises a fresh water inlet, both cold and hot, the water flowed in here meets a recirculation loop comprising a recirculation water inlet, which in fact is one point along the loop before subsequent key units being a filter and then a heater. In the flow path up-streams, the user outlet is provided subsequently to the heater. As seen in FIG. 1 there is arranged a drain being a point from which water is either wasted via the water recirculation outlet or sent to recirculation. As said above, to perform the method according to the present invention, the opening of valves (not shown) may be used. Moreover, sensors providing data to the processing unit and also a pump providing recirculating force are other units not shown in FIG. 1.

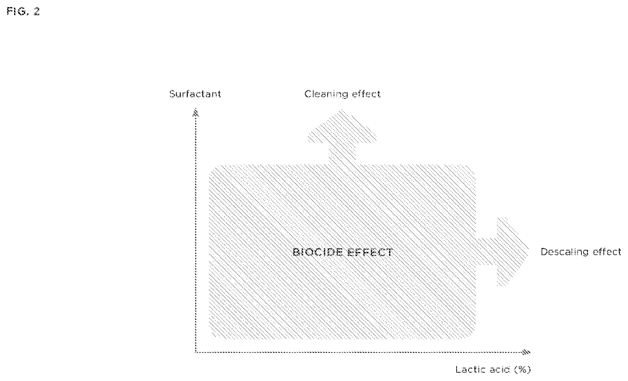

In FIG. 2 there is shown a schematic view of the disinfecting (descaling) effect and cleaning effect when using lactic acid as the disinfecting agent and a surfactant as the cleaning agent according to the present invention.

1. A method for disinfection and cleaning of at least one part of a device intended for recycling of water, said method involving flowing lactic acid, citric acid and/or an enzyme based or surfactant based solution into the part intended to receive the disinfection and cleaning.

2. The method according to claim 1, wherein the method involves at least flowing lactic acid into the part intended to receive disinfection treatment.

3. The method according to claim 1, wherein the method involves flowing both an antibacterial agent for the disinfection and also an antifouling agent for the cleaning into the part intended to receive the disinfection and cleaning.

4. The method according to claim 1, wherein the device comprises a flow path for recycled water, a fresh water inlet, a recirculation water inlet and a recirculation water outlet, a user outlet, a pump, sensors, valves, a heater and a filter and wherein the method involves flowing lactic acid, citric acid and/or an enzyme based or surfactant based solution into at least part of the flow path for recycled water.

5. The method according to claim 4, wherein the user outlet is one part being disinfected and cleaned.

6. The method according to claim 5, wherein the device intended for usage and recycling of water is a recirculating shower.

7. The method according to claim 5, wherein the process order subsequent to the filter is the heater and then finally the user outlet and wherein flowing of lactic acid, citric acid and/or an enzyme based or surfactant based solution is performed into at least the user outlet of the device.

8. The method according to claim 7, wherein the flowing of lactic acid, citric acid and/or an enzyme based or surfactant based solution is provided from the filter into the system and thus into all of the system arranged subsequent to the filter, also comprising the heater and the user outlet.

9. The method according to claim 8, wherein a disposable filter unit is replaced with a cartridge containing the lactic acid, citric acid and/or an enzyme based or surfactant based solution for allowing for performing the disinfection treatment.

10. The method according to claim 8, wherein the filter is a disposable filter unit also comprising a dosing capsule containing lactic acid, citric acid and/or an enzyme based or surfactant based solution and wherein the dosing capsule discharges the content when the filter is pressurized.

11. The method according to claim 4, wherein the lactic acid, citric acid and/or an enzyme based or surfactant based solution is provided in a dosage container which content is poured into a drainage and thus into a recirculation loop to be flowed into the filter, the heater and the user outlet.
